(19) United States
(12) Patent Application Publication
(10) Pub. No.: US 2026/0226419 A1
(43) Pub. Date: Aug. 6, 2026

(54) METHOD FOR THE ISOLATION OF ADIPOSE-DERIVED STEM CELLS FROM TISSUE BIOPSIES

(71) Applicant: Cultimate Foods GmbH, Berlin (DE)

(72) Inventors: Mariia Abyzova, Robert-Rössle-Straße 10 (DE); Hanna Marline Kirsch, Berlin (DE); Simon Zschieschang, Berlin (DE); Jordi Morales Dalmau, Berlin (DE)

(73) Assignee: Cultimate Foods GmbH, Berlin (DE)

(21) Appl. No.: 19/532,084

(22) Filed: Feb. 6, 2026

Related U.S. Application Data
(60) Provisional application No. 63/754,616, filed on Feb. 6, 2025.

(51) Int. Cl. C12N 5/0775; A23L 13/00; C12N 5/00

(52) U.S. Cl. CPC C12N 5/0667; A23L 13/00; C12N 5/0087; C12N 5/562; C12N 2500/02

(57) ABSTRACT

Isolated adipose-derived stem cells and methods of isolating the stem cells from the adipose tissue are disclosed. The isolated stem cells possess benefits relative to prior art methods in that there is better purity, better yield, shorter processing time, and/or other superior optimization values. The method comprises three main steps: 1) tissue sampling and tissue preprocessing, 2) digestion process of the tissue and cell isolation, and 3) cell cultivation and growth, and the parameters in each of these three main steps have been adjusted to provide the optimal isolation of the stem cells.

This application claims priority under 35 USC 119(e) to US Provisional Application No. 63/754,616 filed February 6, 2025, the entire contents of which are hereby incorporated by reference in its entirety.

### FIELD OF THE INVENTION

The present invention relates to isolation methods of stem cells from adipose tissues, especially in the application of fabricating cell-cultivated products. The different parameters to improve the isolation yield and other parameters related to the isolation are evaluated herein to generate the highest quality of the cells.

### BACKGROUND OF THE INVENTION

According to the Food and Agriculture Organization (FAO), the demand for meat will increase by up to 70% by 2050, which will be an immense challenge for the livestock system. The ethical issues surrounding meat consumption include concerns about animal welfare, environmental sustainability, and the health impact on both animals and humans. Plant-based, precision fermentation and cell-cultivated technologies are the most relevant sustainable and ethical alternatives to traditional meat, with a projection of $90tn by 2040. To make them a reality, it is essential to achieve taste and price parity, guided by innovation and consumer adoption.

The production of cell-cultivated products is a complex process, typically divided into four main parts: cell isolation, cell proliferation, cell differentiation and final product formulation. Although these parts have defined processes and goals, they are not independent of each other. For instance, the final product will depend on the quality of the differentiation process and quantity of cells during the proliferation; at the same time, these will directly depend on the characteristics of the bioprocess, and media formulation, which are adapted to the cells used in the process. Therefore, the quality of the cells obtained during the isolation influences the rest of the processes. It is of utmost relevance to define an isolation method, in which the quantity of the obtained cells and their quality is optimized.

Since the first time cell-cultivated meat products were reported in 2002, a large number of processes to isolate cells have been developed. Although this helped in the adoption of this field around the world, it created diverse non-standardized isolation processes. One of the drawbacks of the currently available process is the lack of standardization of an isolation process with very relevant yields, and characterization of the cells for cell-cultivated products.

In this invention, the most relevant parameters are defined to isolate adipose-derived stem cells, including the extraction location of the sample, the composition of the digestion buffer, temperature, oxygen concentration, and time and speed used during the digestion as well as the first culture media after the digestion process, and their dependence on the number of isolated cells, and other parameters related to the quality of the cells.

### BRIEF SUMMARY OF THE INVENTION

The present invention is related to the definition and optimization of the parameters for the isolation of adipose-derived stem cells. The invention achieves cell yields of more than 1,500,000 cells per gram of isolated tissue after less than 24 hours after isolation. In a variation, the minimum yield of cells by following the methodology of the present invention is at least 500,000 cells, or alternatively, at least 750,000, or alternatively, at least 1,000,000, or alternatively at least 1,250,000 cells in a 24 hour period. This/these yield(s) is/are unexpectedly superior to the yields that are seen in prior art protocols from extracted tissue.

Thus, the present invention relates to isolated stem cells from adipose tissue and methods of isolating the stem cells. The isolated stem cells possess benefits relative to stem cells isolated by known methods in that there is a higher purity, higher yields, shorter processing time, and/or other superior optimization values. The method comprises three main steps: 1) tissue sampling and tissue preprocessing, 2) digestion process of the tissue and cell isolation, and 3) cell cultivation and creation of the master bank, and the parameters in each of these three main steps have been adjusted to provide the optimal isolation of the stem cells.

### BRIEF DESCRIPTION OF THE SEVERAL VIEWS OF THE DRAWING

FIG. 1 shows a block diagram illustrating the process between sampling fat tissue from an animal to the midway step in the process.

FIG. 2 shows a block diagram illustrating the process between the midway step at the end of FIG. 1 to the isolation of the stem cells from the adipose tissue.

FIG. 3A shows the relative amounts of cells produced from different areas of the animal using a normalized isolation yield of porcine adipose-derived stem cell isolation from two to twelve donor animals using four different tissue sampling locations.

FIG. 3B shows the confluence of cells in P0 isolated from different body parts of the animal, wherein the animal is an isolated porcine adipose-derived stem cells originating from four different sampling locations of three different donor animals, 24h after isolation.

FIG. 4 shows the confluence of cell cultured with different glucose and oxygen concentrations.

FIG. 5A shows the effect of different ascorbic acid concentrations on proliferating cells as box plot diagram of cells/field of view after three days of cultivation of two adipose-derived stem cell lines in cell culture media supplemented with different concentrations of FGF2 (FGF) and ascorbic acid-2-phosphate (AA).

FIG. 5B shows a CellTiter Blue Viability assaynormalized to control of three adipose-derived stem cell lines after one or three days of cultivation in cell culture media supplemented with a concentration range of ascorbic acid-2-phosphate.

FIG. 5C Normalized isolation yield of porcine adipose-derived stem cell isolation from four donor animals using three different concentrations of ascorbic acid-2-phosphate during digestion, harvested less than 48h after isolation.

FIG. 6 shows the cumulative population doublings (CPD) of the cells digested with and without ascorbic acid under FIG. 6A normoxia or under FIG. 6B, hypoxia.

FIG. 7 shows a cytotoxicity test of GMS over four days.

FIG. 8A shows the cell yield of the isolation (cells/g of tissue) depending on the GMS concentrations during the digestion process.

FIG. 8B shows pictures with cell confluence of cells from two different animals (8, 10) digested with increasing GMS concentrations from top to bottom.

FIG. 9 Normalized isolation yield of porcine adipose-derived stem cell isolation from three to six donor animals using four different concentrations of glycerol monostearate during enzymatic digestion.

FIG. 10 Normalized isolation yield of porcine adipose-derived stem cell isolation from four donor animals using three different rotation speeds during digestion, harvested less than 24h after isolation.

FIG. 11A shows the cell confluence less than 24h after isolation after different digestion times.

FIG. 11B shows normalized isolation yield of porcine adipose-derived stem cell isolation from three donor animals using three different enzymatic digestion times, harvested less than 48h after isolation

FIG. 12 shows the cell confluence after the digestion processes under different temperatures.

FIG. 13A shows cell confluence under hypoxia and normoxia.

FIG. 14 shows number of cells per well under different cultivation temperatures.

FIG. 15A shows cells/FOV of cells cultivated with media supplemented with different growth factors.

FIG. 15B shows cells per field of view after three days of cultivation of adipose-derived stem cell line 1 (A) and 2 (B) in cell culture media supplemented with different growth factors.

FIG. 16 shows the cumulative population doublings of cells cultivated in cell culture media supplemented with different growth factors.

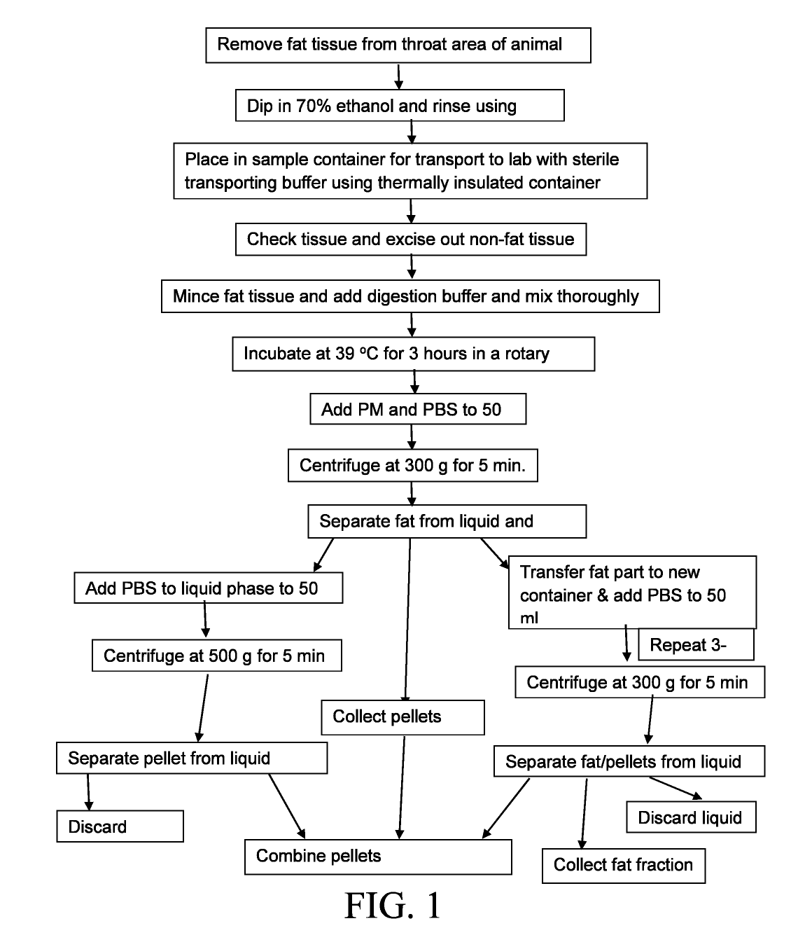

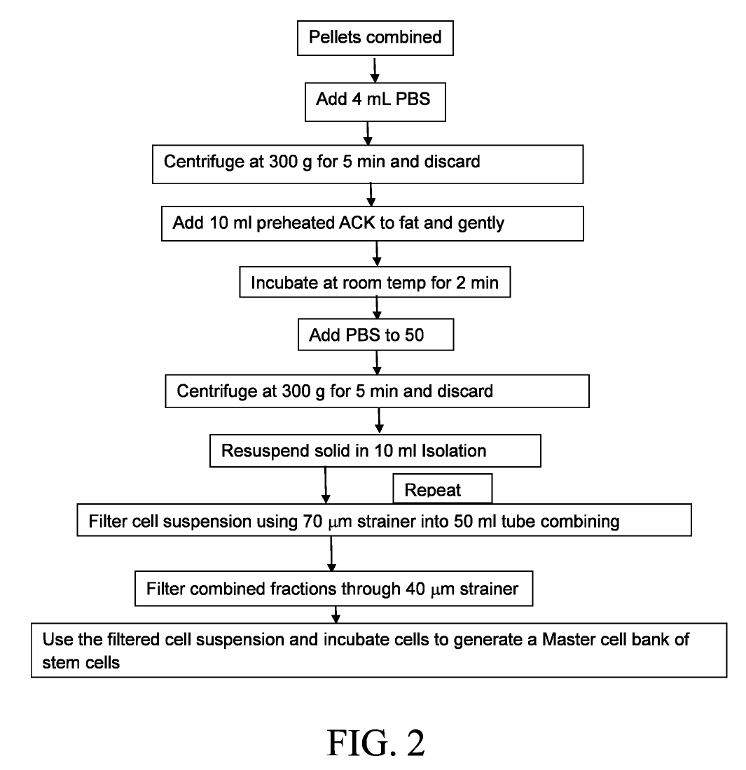

### DETAILED DESCRIPTION OF THE INVENTION

The present invention relates to the isolation of adipose-derived stem cells. In an embodiment, the parameters that are disclosed herein allow for an optimized isolation that is superior to any isolation in the prior art. In an embodiment, there are three main process steps to achieve the isolation of adipose-derived stem-cells. These steps are: 1) tissue sampling and tissue preprocessing, 2) digestion process of the tissue and cell isolation, and 3) cell cultivation and creation of the master bank. The more detailed process is described in the steps listed below.

### I. Tissue sampling and preparation/tissue preprocessing

### Preparation of buffers and solutions in lab

0. Preparation

a. Preparation of transportation buffer (one day before)

b. Store Transportation buffer, PBS, EtOH, and hand sanitizer at +4 °C before placing it in a thermally insulated box.

c. Store the coolants at -20^{o}C.

### Abattoir

1. Cut samples of ~20 g of subcutaneous fat tissue from the throat area of the slaughtered animals directly after slaughter using sterilized scalpel and tweezers.

2. Briefly submerge the fat tissue in 70% ethanol and rinse in sterile PBS after sample excision.

3. Place approximately 20 mL of tissue into a 50 mL conical centrifuge tube filled with approximately 30 mL of sterile transporting buffer. Transporting buffer comprises of 1x PBS, 5 mM D-glucose, 5% antibiotic agent (such as penicillin-streptomycin solution) and 3% antifungal agent (such as amphotericin B solution).

4. Transport the tissue from the abattoir to the laboratory in a thermally insulated container. The time between sample excision and commencing the procedure in the laboratory should be kept to a minimum and if possible, it should be started within one hour after sample excision.

### Laboratory

5. Weigh empty 50 mL conical centrifuge tubes and record the weights.

6. If present in the sample, remove any non-fat tissue (such as muscle tissue, connective tissue or blood vessels) under a laminar flow cabinet.

7. Put approximately 20 g of fat tissue of each sample into individual 50 mL conical centrifuge tubes.

a. Mince the tissue with a device such as scissors or scalpel until homogeneity.

b. Distribute the minced tissue evenly between two different 50 mL conical centrifuge tubes and take record of the weights.

c. Equalize weights as accurately as possible (+/- 0.06 g) between the 50 mL conical centrifuge tubes of the sample.

### II. Digestion process

8. Add 0.5 mL of digestion buffer (Hanks’ Balanced Salt Solution with 1% (w/v) dispase II and 1% (w/v) collagenase I) per 1 g of tissue to the homogenized tissue, and invert 50 mL conical centrifuge tubes multiple times.

9. Incubate the digestion mixture in the incubator at 39 °C (porcine body temperature) for 3h, in a rotary mixer at 30 rpm.

10. After digestion process incubation:

a. Add 10 mL of proliferation medium and fill the 50 mL conical centrifuge tube up to 50 mL with phosphate buffered saline (PBS). Proliferation media comprises of DMEM/F12, 10% FBS, 2 mM L-glutamine, 0,2% primocin, and FGF 2 ng/mL,

b. Centrifuge at 300 g for 5 min at about 10^{o}C or alternatively, from about 4-10°C

11. Transfer the whole fat fraction from the top of the 50 mL conical centrifuge tube to a new 50 mL conical centrifuge tube. Because there may not be a clear delineation between the fat fraction and liquid fraction and because of the difficulties of removing “softer” fat, one might more easily effectuate transfers if the fat is a bit “harder”, which can be achieved by placing the centrifuged sample in a refrigerator (~2-5 ^{o}C) for about three minutes.

12. Distribute the liquid fraction into multiple 50 mL conical centrifuge tubes, add each 50 mL conical centrifuge tube up to 50 mL with PBS and centrifuge at 300 g for 5 min. Discard the supernatant.

13. Resuspend and combine all cell pellets (pellets of the initial centrifugation, liquid and fat fractions) isolated from the same animal into a new 50 mL conical centrifuge tube in 6 mL of PBS.

14. Rinse all tubes of one animal with 4 mL PBS, add it to suspension, and centrifuge for 5 min at 300 g. Discard supernatant.

15. For all tubes perform erythrocyte lysis using the steps listed below:

a. add 10 mL of preheated Ammonium-Chloride-Potassium lysis buffer (ACK) and resuspend gently

b. incubate the mixture for 2 min at room temperature

c. add PBS up to 50 mL

d. centrifuge 5 min at 300 g

e. discard supernatant and keep pellets

### III. Cell cultivation and creation of the master cell bank

16. Resuspend each pellet in 25 mL of isolation medium. Isolation medium comprises of DMEM/F12, 10% FBS, 2 mM L-glutamine, 0,2% primocin, and FGF2 ng/mL.

17.Filter the cell suspension through a 70 µm strainer into a 50 mL conical centrifuge tube. Subsequently filter through a 40 µm strainer into a T175 cell culture flask.

18. Incubate the cells overnight (at least 12-18 h) at 39°C, then change the media to fresh isolation medium.

19. Exchange media every second to third day.

20. Once the cells reach 70-80% confluence, detach cells and characterize (ISCT criteria) and cryopreserve cells in a “master cell bank”.

From the above, it should be apparent that the entire cell isolation process can be divided into three main steps. The three steps all provide opportunities for optimization. Optimization takes place in the areas of 1) tissue sampling and preparation/tissue preprocessing, 2) the digestion process and the subsequent 3) cultivation of the cells up to the creation of the master cell bank.

Various parameters were explored to ascertain what parameters can be varied to provide optimization of the process (e.g. best purity, highest yield, shortest processing time, and other optimization values).

Several important parameters were studied and experiments were conducted to find what values for these parameters provide the adipose-derived stem cells that attained the highest yields and/or resulted in cells with the best cell quality. The parameters studied at the different process steps included:

### 1. Tissue sampling and preprocessing

Tissue sampling and preprocessing describes the acquisition of the animal tissue, its transport from the abattoir to the laboratory, as well as its preparation for the enzymatic digestion under sterile conditions, including non-fat tissue removal and mincing of the tissue.

### Breeds, Sex and Age

During the tissue sampling process different animals at different ages and different sexes were evaluated. The preliminary data suggests that the group of animals that were 1-3 months old were the preferred animal group relative to the animals that were 5-6 months old due to higher yields. However, adequate yields were later found in both tested groups.

Therefore, it was discovered that irrespective of the individual (different animals, age 1-3 or 5-6 months - different breeds, sex, etc.) from which tissue was excised, (for example, for the porcine species such as Duroc, Pietrain and Landrace, and either male and/or female) the above procedure gave good results for all breeds tested. All males were castrated and/or neutered. The described protocol can be applied in stem cells isolations for all mammalian species.

### Animal Status

(alive/ex-vivo/dead)

Much of the data that was obtained was from ex-vivo animals. However, from testing, the results that are obtained are reproducible with biopsied tissue from living animals.

### Sampling Location

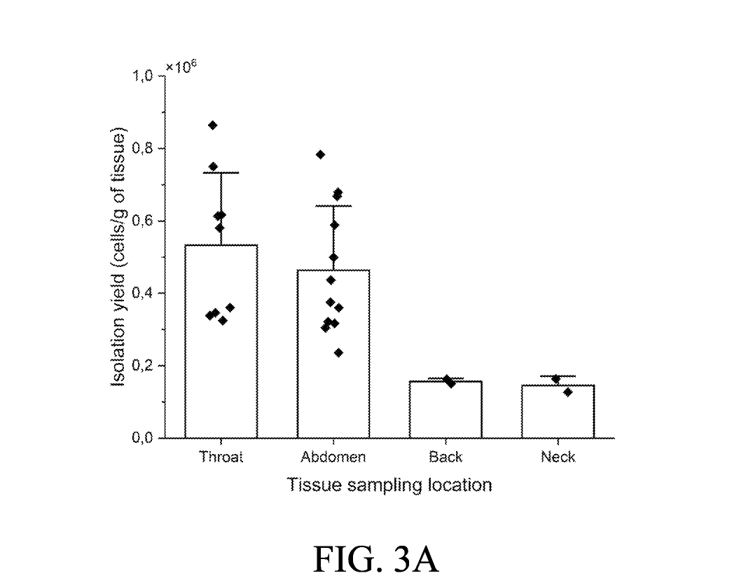

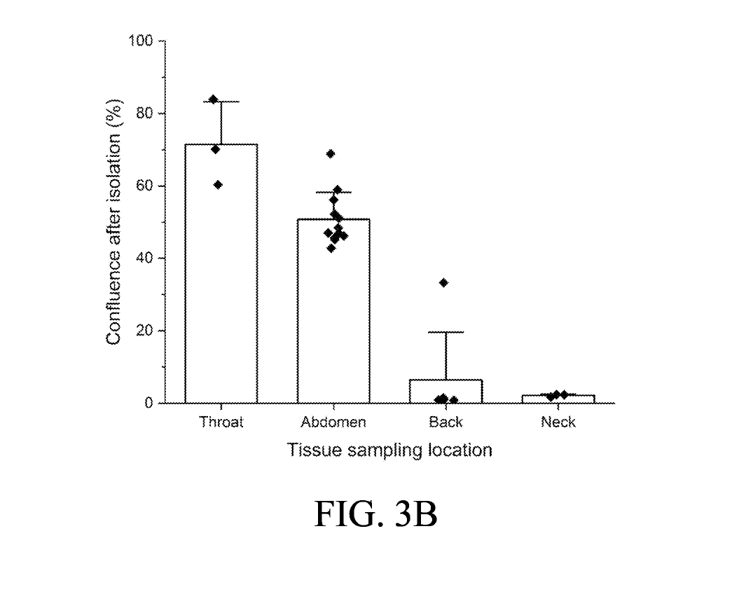

The adipose tissue was sampled from different anatomic locations of the animals. The growth, proliferation and differentiation of the isolated cells were analyzed from each of the tested locations to see which areas yielded the best results. The amount of isolated cells per gram of extracted tissue at various locations was calculated (see FIG. 3A) and the differentiation capacity of the cells was analyzed. The amount of cells isolated from each location was measured via the confluence of the cells one day after isolation (after media exchange) (See FIG. 3B). It was found that the tissue extracted from near the throat area yielded the best results (see FIG. 3).

### Treatment and Transport Conditions Abattoir-Laboratory

The treatment and transport of samples, both within the abattoir and during transfer to the laboratory, are critical determinants of the quality of the isolated cells. The processing in the laboratory should be started immediately after excision, or alternatively, within one hour after sample excision up to 48 hours after excision. For transport, it is recommended to maintain the tissue samples within a temperature range of 2^{o}C to 45^{o}C or alternatively, 4-20°C. Various sterilization methods, antibiotics and antimycotics (e.g. primocin) may be employed.

The optimized conditions were incorporated into the process enumerated supra.

### 2. Digestion process

The digestion process describes the method to liberate cells from the extracellular matrix (ECM) of the previously acquired and transported cells (in I. Tissue sampling and preprocessing) using collagen-degrading enzymes.

Various factors play a role in the digestion process, and the various digestion process parameters were evaluated. Currently, the primary cell isolation processes of the prior art rely on mechanical disruption of the tissue followed by a digestion of ECM-proteins (extracellular matrix-proteins) with enzymes, such as collagenase. It was found that the composition of the digestion buffer has one of the largest effects on the efficiency of the isolation. The use of appropriate digestion enzymes and other components ensures that the native collagen in the animal tissue is degraded and that the individual cells can be effectively isolated from the tissue.

Accordingly, to achieve a highly efficient digestion process, there are competing factors that affect the efficiency of the process. For example, one desires to achieve accessibility of the enzymes to the proteins using an optimized temperature, while other factors, such as the proper level of calcium ions, ensure the stability of enzymes as well as their activity. The accessibility of the enzymes to the proteins is very important but has to be balanced with the corresponding cell quality and viability of the cells. These trade-offs were all considered, and the enumerated procedure referenced supra resulted in a successful isolation process. The results of the parameters of the digestion process that were optimized follow:

### Enzymes

Enzymes are an important component of a digestion buffer because they catalyze the targeted degradation of extracellular matrix structures that maintain tissue integrity and enable a controlled dissociation of the tissue to release viable and structurally intact cells while minimizing mechanical stress and preserving phenotypic markers. The recommended concentration range is between 0.01-4% (w/v) of at least one collagen-degrading enzyme, such as dispase and collagenase I (e.g. Gibco Dispase II and Collagenase Type I Powder).

It was discovered that the use of dispase II, and collagenase I at a concentration of 0.3-1% gave ideal results.

### Calcium Chloride/Calcium Ions

Collagen-degrading enzymes, such as collagenase I, are calcium-dependent metalloproteases. Therefore, the appropriate range of calcium ions can improve the activity of the collagen-degrading enzymes, liberating more cells from the extracellular matrix of the fat tissue. Depending on the basal solution used for the digestion, the addition of 0-20 mM calcium chloride in the digestion buffer is recommended. It is believed that calcium ion concentrations at levels between about 2 mM up to 10 mM increase the enzymatic activity and give ideal results.

### Glucose Concentration

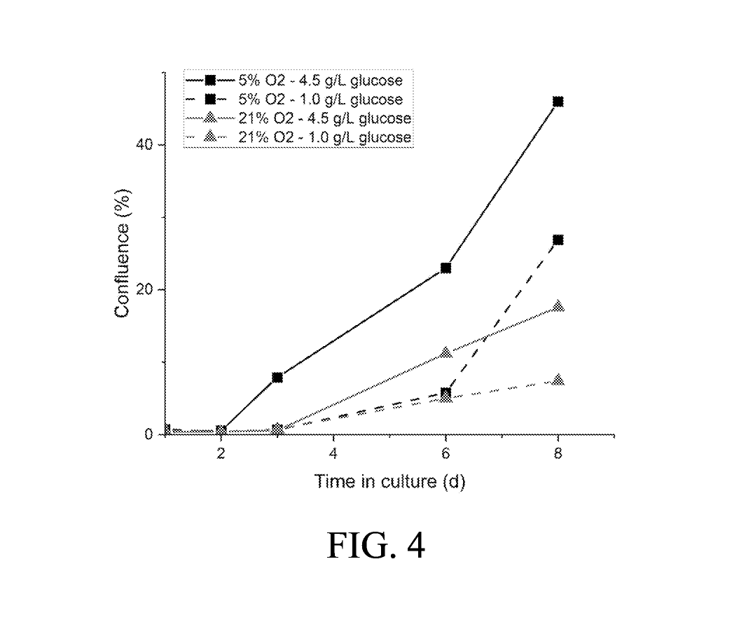

It was discovered that the ideal glucose concentration depends on the media and the oxygen concentration used during cell cultivation. The results of testing showed that higher glucose concentrations in the media improved the cell growth even more when cultured in hypoxia (See FIG. 4). Thus, it should be apparent that there is a benefit to measuring oxygen concentrations and using the oxygen level with the appropriate glucose amount to get the best results. For example, it might prove to be advantageous to deprive the cells of oxygen and combine the hypoxic conditions with high glucose levels to give the best results. In a variation, the range of glucose can be from 0.01 to 100 mM, or alternatively, 60 mM to 100 mM glucose. It should be understood that an amount that is about 25 mM is considered a high glucose level and 50 mM is considered to be a very high glucose level. Alternatively, these agents can be omitted. However, if this step is omitted it is expected that the yields will be lower.

### Ascorbic Acid

In addition to its antioxidant properties, ascorbic acid-2-phosphate (AA) can promote proliferation, reduce apoptosis and increase oxidative respiration and glycolysis in cells.

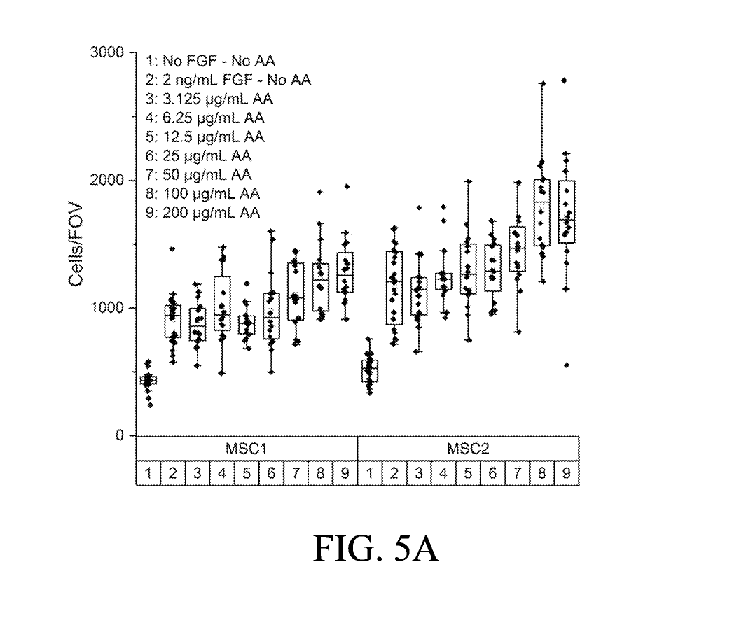

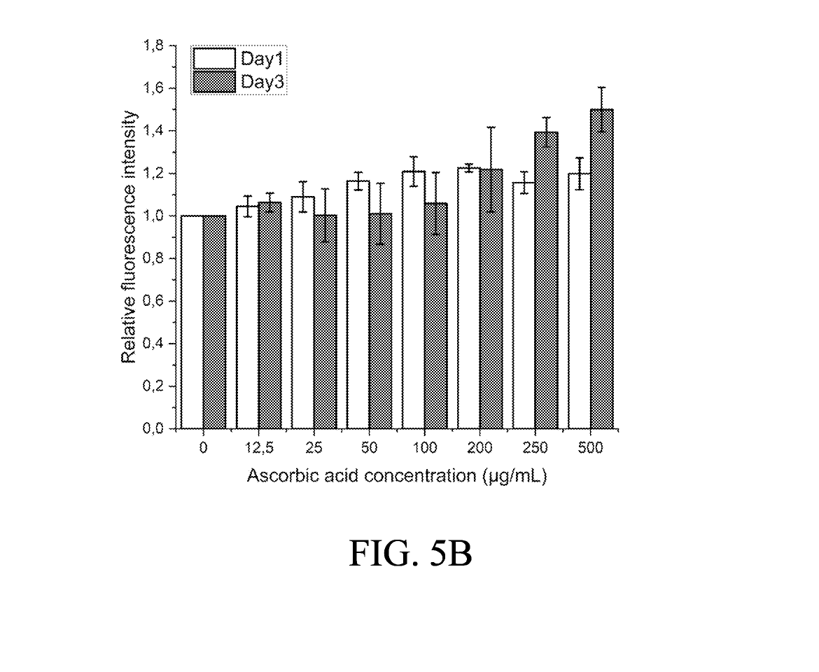

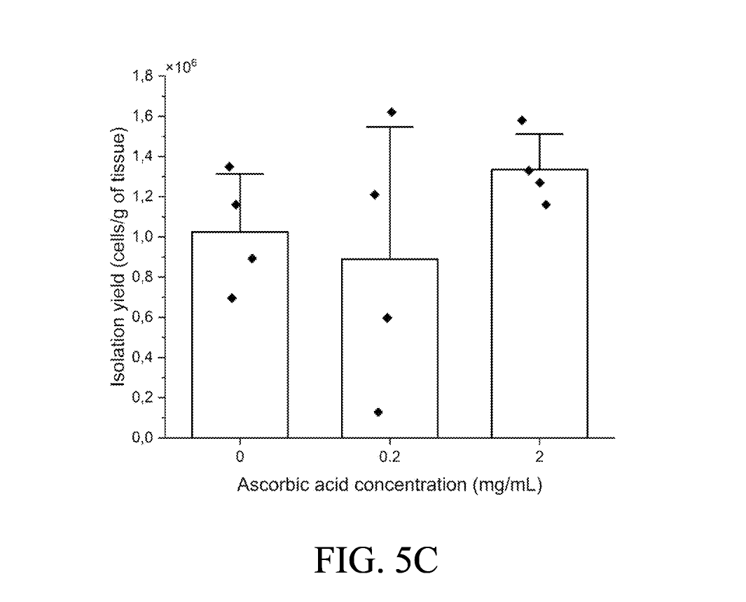

In preliminary experiments, the optimal AA concentrations for the cell cultures were determined and subsequently used as one of the concentrations in the digestion buffer (FIG. 5A and B).

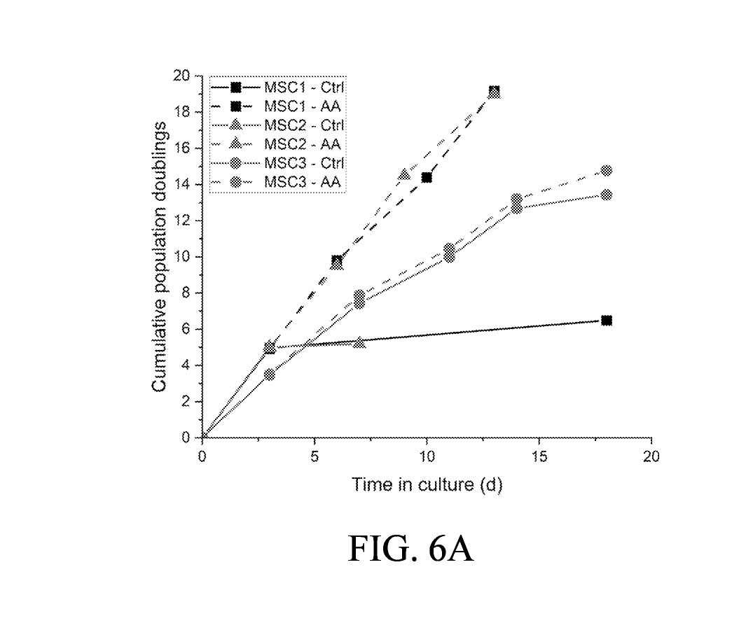

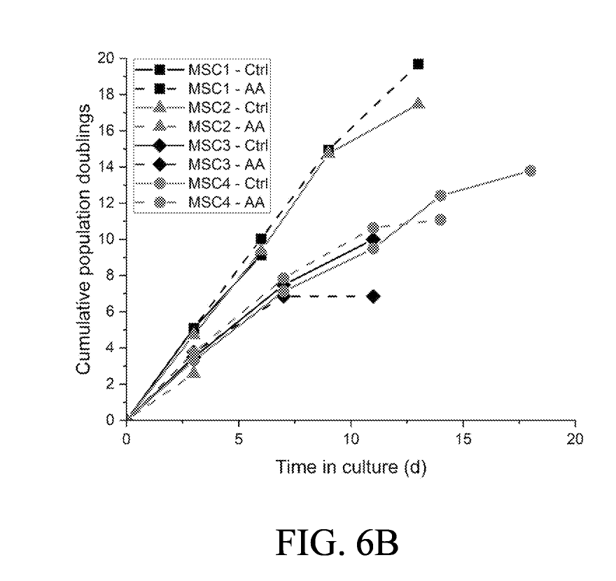

The digestion buffer contains AA at a concentration within the recommended range of 200 to 2000 µg/mL, with higher concentrations (e.g. 2000 µg/mL) demonstrating a positive effect on the cell yield (cells per gram of tissue) (FIG. 5C). The addition of AA to the digestion buffer ensures longer and faster proliferation of the cells (higher cell population doublings) under normoxic conditions (usually around 20-21% oxygen) (FIG. 6A).

Although the addition of AA gave good results under normoxic conditions, the same results were not observed under hypoxic conditions (FIG. 6B). Thus, to achieve the best results, one might consider cultivating under normoxic conditions (FIG. 6). While various ascorbic acid derivatives can be used, 2-phospho-ascorbic acid is recommended due to its stability in cell culture media (e.g. 2-Phospho-L-ascorbic acid trisodium salt from Sigma). Alternatively, these agents can be omitted. However, if this step is omitted it is expected that the yields will be lower.

### Glycerol Monostearate

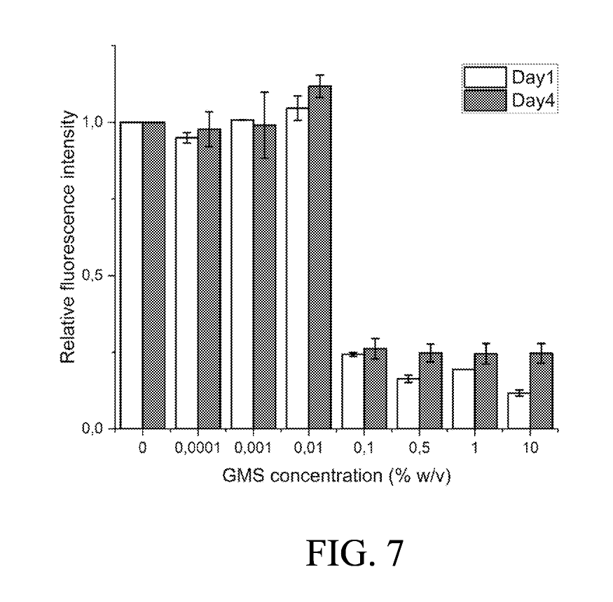

The effect of glycerol monostearate (GMS) was evaluated. In addition to successfully minced tissue, enzyme concentration and activity are crucial for efficient cell isolation. However, isolating cells from hydrophobic adipose tissue with enzymes in a hydrophilic solution may result in active enzymes that do not have access to the corresponding proteins of the ECM. Therefore, without being bound by theory, it is assumed that emulsifiers and amphiphilic agents can be used to optimize the access of enzymes to the ECM proteins. An amphiphilic agent that increases enzymatic access to hydrophobic extracellular matrix is for instance GMS. A commonly used food additive, which is a monoglyceride that is widely used as an emulsifier in the food and pharmaceutical industries. Initially, the cytotoxicity of GMS on 2D cell monolayers was tested over several days (FIG. 7). Compared to the control (0%, without GMS), no cytotoxic effect of GMS was seen up to a concentration of 0.01% (w/w). A concentration of 0.01% even showed a slightly higher cell viability compared to the control.

At higher concentrations (above 0.1 % (w/w)), the GMS-containing medium formed a creamy layer on the top of the cell monolayer after a few hours. The blocking of cell metabolism and oxygen exchange could also lead to cell death and is not necessarily a direct consequence of the cytotoxicity of GMS. However, as the GMS only interacts with the tissue for 3 hours during digestion, the risk of a comparable effect of GMS on the cells in the digestive buffer is very low.

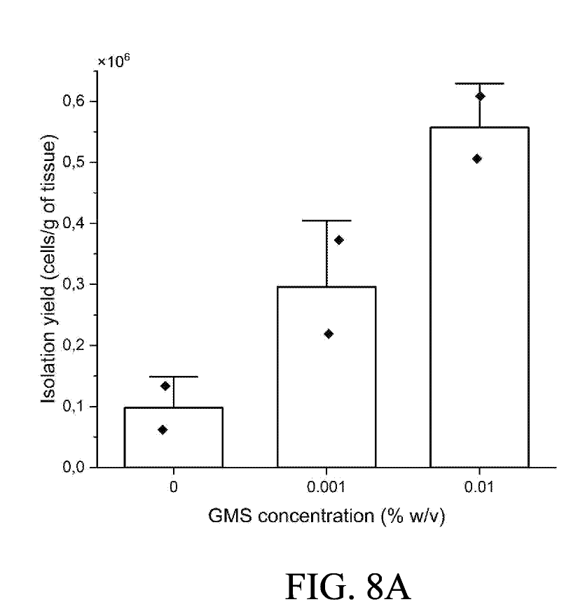

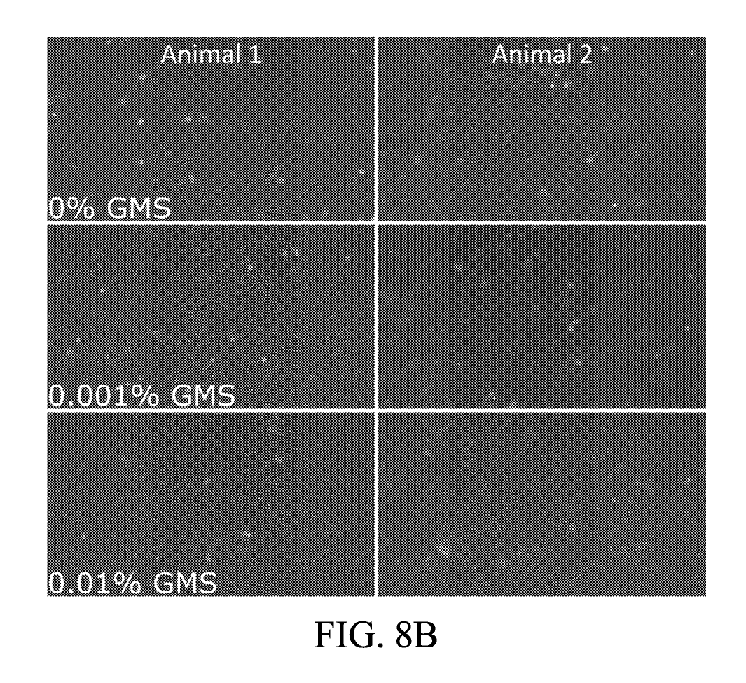

An initial preliminary experiment demonstrated that cell confluence and cell yield (cells/g tissue) increases with increasing GMS concentration in the digestion buffer (FIG. 8).

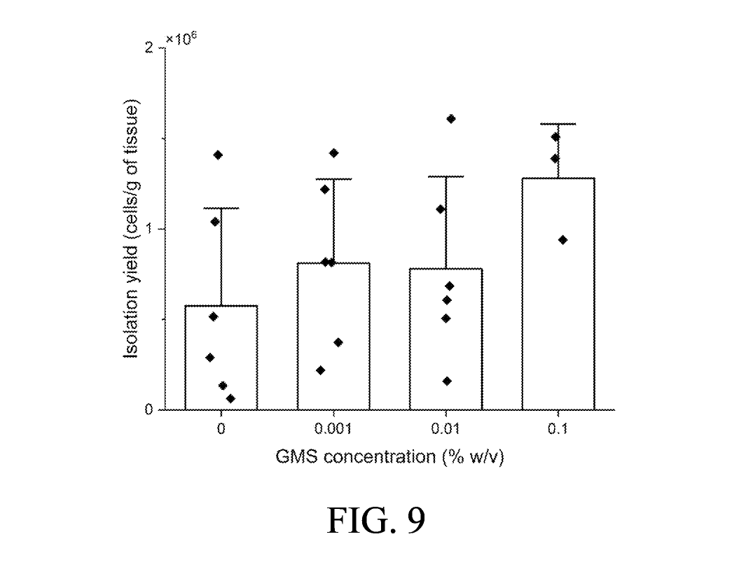

After additional isolation parameters had been optimized, a subsequent experiment involving four animals and three different GMS concentrations confirmed that GMS in the digestion buffer can increase the cell yield per gram of tissue (results combined in FIG. 9).

Thus, it appears that there is an optimal level of GMS to achieve the best cell yield, while simultaneously avoiding excessive cell death. Good results are seen for GMS levels between 0.001% and 1% (w/w). It should be noted that a concentration range of up to 10% of the amphiphilic agents such as GMS is recommended for inclusion in the digestion buffer to increase the resulting cell yield and cell quality. In a variation, one or more amphiphilic agents that increase enzymatic access to hydrophobic extracellular matrix is desired. Alternatively, these agents can be omitted. However, if this step is omitted it is expected that the yields will be lower.

### Rotation Speed

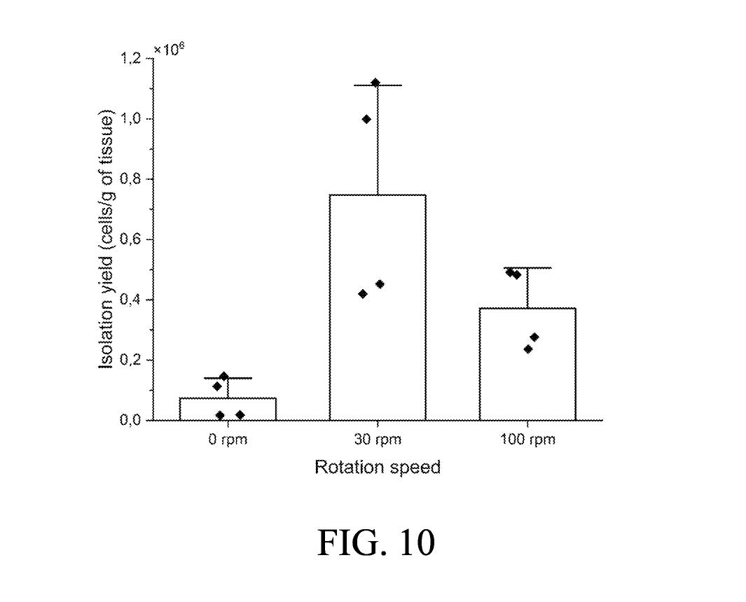

The type and speed of shaking was evaluated. Mechanical agitation using an orbital or rotary shaker is commonly used during enzymatic tissue digestion to ensure uniform exposure of the sample to the digestion enzymes. Continuous, controlled shaking enhances enzyme-tissue contact, improves diffusion and prevents sedimentation of tissue fragments, thereby promoting a more efficient and reproducible dissociation process. Different devices differ by form of shaking (e.g. rotary, orbital), shaking amplitudes, orbital diameter, vibration pattern and mixing intensity, which can introduce distinct mechanical forces during enzymatic digestion, potentially altering for instance the enzyme-tissue contact and shear stress for the cells. This may influence both the efficiency and consistency of tissue dissociation. To ensure optimal mixing during enzymatic digestion, the use of an alternative device to an orbital shaker - such as a rotary shaker - is recommended. For effective enzymatic digestion, a rotation speed of 30 rpm is recommended (FIG. 10). However, it should be understood that rotation or agitation speeds between no agitation to a rotation speed of 300 rpm can be used.

### Digestion Time

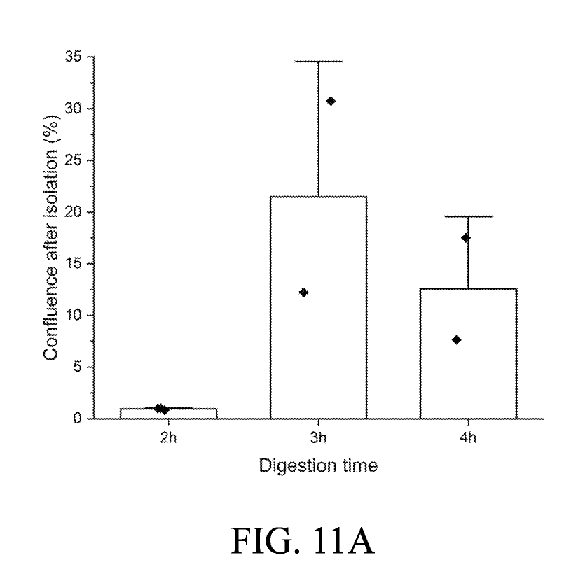

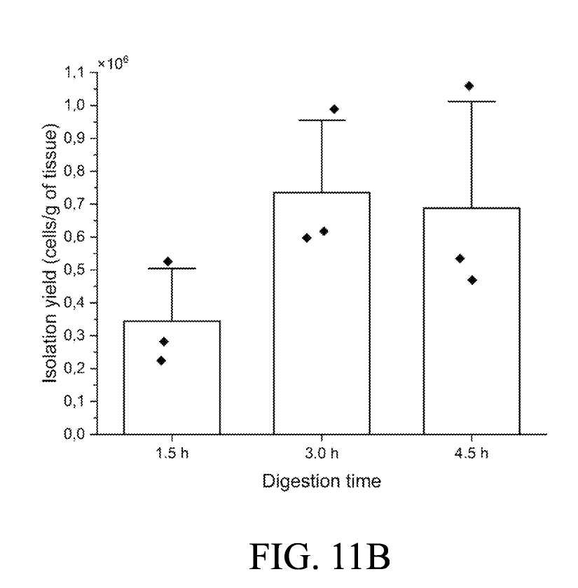

To achieve optimal tissue digestion, it is not only the enzyme concentration and temperature that are important. The more time the enzymes have to access the ECM proteins or to break down more proteins (such as collagen), the better the tissue can be digested. However, some other competing factors must be considered that can affect cell quality and quantity during the digestion process. Individual cells that are not protected by an ECM can be more easily damaged by shear stress or oxidative radicals, etc. during the digestion process. The resulting dead cells can release factors that negatively impact the viability of other cells. It is therefore important to optimize the timing of digestion to achieve an optimal yield of isolated single cells with maximum cell quality (viability, stability, etc.). Two independent experiments demonstrated that digestion times between 3 and 4.5 hours is superior (higher cell confluence and increased cell yield (cells/g tissue) to digestion times of 2 hours or less (FIG. 11A and 11B). Depending on the collagenase-degrading enzyme(s), their concentration, and the agitation speed used during the digestion, a digestion time of 20 min up to 24 h is recommended.

### Digestion Temperature

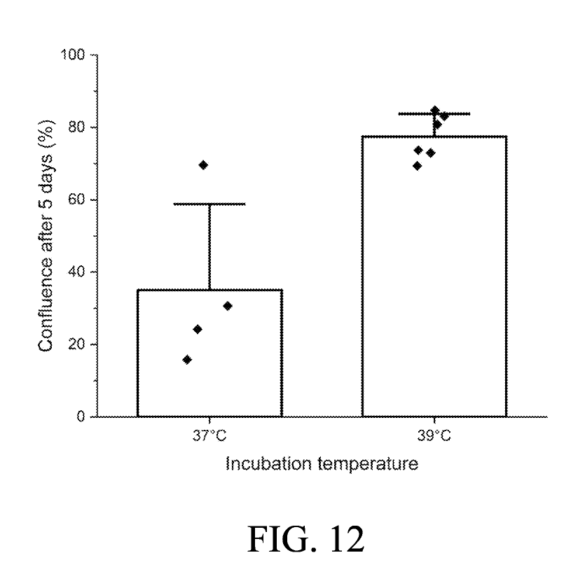

The temperature at which digestion occurred was evaluated. Current isolation protocols for the isolation of primary cells from human or mammalian tissue generally use the optimal temperature for maximum enzyme activity (for collagenase 35-37 °C). Since most mammalian cells are grown at 37 °C, most protocols use 37 °C for the digestion process. However, the present experiments considered the body temperature of pigs (39 °C) and the influence of higher temperatures on adipose tissue (the tissue becomes less viscous). A comparison between 37 °C and 39 °C was performed for the digestion process (FIG. 12).

It was found that the isolation results were better at 39^{o}C.

### Cell cultivation until Master cell bank creation

The cell cultivation until the master bank creation describes the process of treating the cells previously liberated from the ECM of the animal tissue (during II. Digestion process) and culturing them using cell culture techniques until the cells become confluent and are cryopreserved to create a master cell bank.

### Erythrocyte Lysate

The parameters that deal with lysing erythrocytes were evaluated. The buffer used for erythrocyte lysis is commercially available and used in primary isolation protocols (e.g. ACK lysing buffer from Gibco). It is usually used to lyse and remove erythrocytes which can have a toxic effect on stem cells during the isolation and until the first media exchange (as erythrocytes are an abundant cell type that can release hemoglobin, which may lead to the formation of reactive oxygen species (ROS), which may have a toxic effect on the isolated target cells, causing oxidative stress and DNA damage). In the testing, the addition of the lysing buffer did not show any harm or improvement of the cell viability or the cell yield directly after isolation. However, to reduce the risk of toxic effects from erythrocytes, erythrocyte lysate was added to the standard protocol. However, it should be understood that this step may be omitted or performed up to 3 times.

### Oxygen Concentration

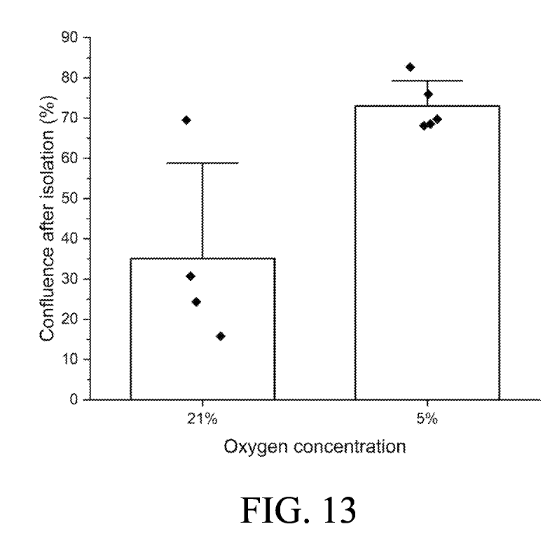

Oxygen is essential in mammalian cell culture because it regulates energy production, metabolic activity and cell survival, making controlled oxygen levels crucial for maintaining healthy and stable cultures. The physiological oxygen concentration varies depending on the type of tissue in the mammalian body. With a few exceptions, most tissues are hypoxic (4-8% oxygen). Therefore, the effects of normoxia (up to 21% oxygen) and hypoxia (0-10% oxygen) on cell proliferation after digestion were investigated (FIG. 13).

### Cell Cultivation Temperature

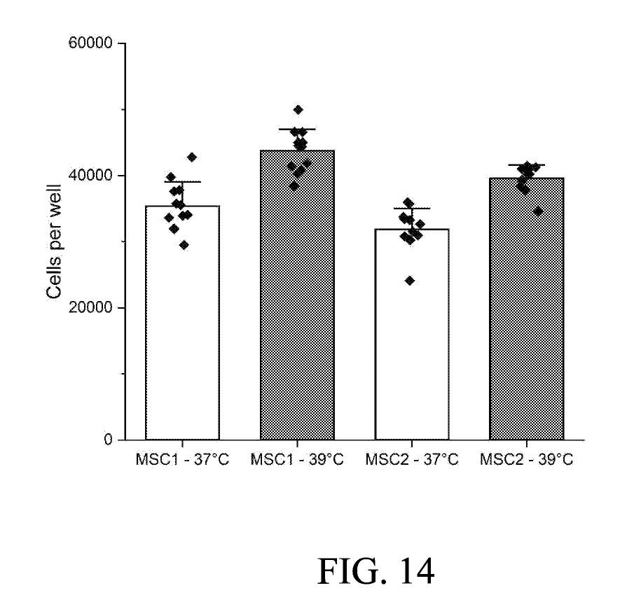

As mentioned in the section on digestion temperature, digestion was performed at 39 °C. Since the body temperature in pigs is higher than in humans, the data from cells cultured at 37 °C and 39 °C was compared (FIG. 14). Primary isolated stem cells show a significant increase in the number of cells/g tissue when cultured at 39°C (after 48 h). It should be noted that the cells can be cultured in a range of temperatures from about 35^{o}C to 42^{o}C.

### Antibiotics

The condition and degree of contamination of biopsies and tissue sampled from donor animals is crucial for the success of an isolation and depends on many factors. These factors include not only the cleanliness of the environment (sampling in rooms other than the collection of gastrointestinal organs), but also the preparation of donor animals (hair removal and fire sterilization of knives and the animal body). It was found that sterilization had a major influence on the probability of contamination. In addition, it was found that the use of antibiotics and antimycotics in the transport buffer was helpful. Accordingly, an antibiotic cocktail might generally be used until the first master cell bank is cryopreserved. However, due to the sterilized preparation of the animals, the sampling procedure employed, and the composition of the transport buffer, primary cells were obtained that were completely contamination-free. It was found that due to the cleanliness (and diligence) of the sampling procedure, antibiotics were not needed after the digestion process.

Primocin was an antibiotic that was tested and it gave good results in the isolation media in general protocol, but it was discovered that it was not needed after the digestion process as the cells after the digestion process were essentially contamination-free. If antimicrobial supplementation is nevertheless required, a concentration range of 0 to 300 µg/ml of antibiotic or antimycotic substances, such as primocin is recommended, with 100 µg/ml providing optimal performance for the application of primocin. Although Primocin was tested, it should be recognized that other appropriate antibiotics are contemplated and may be used.

### Growth Factors

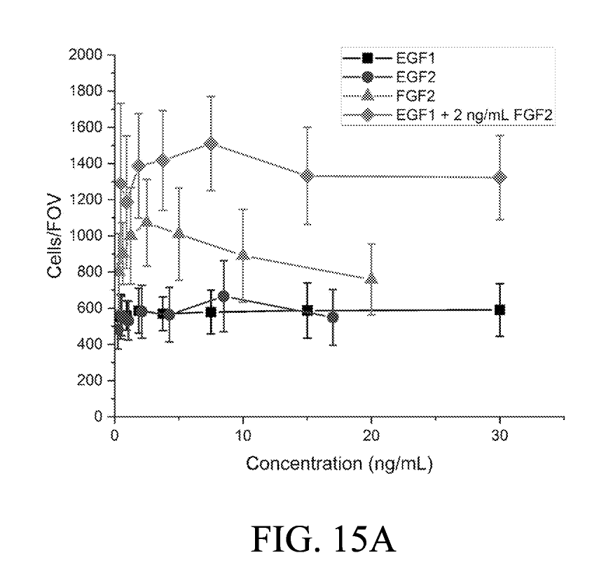

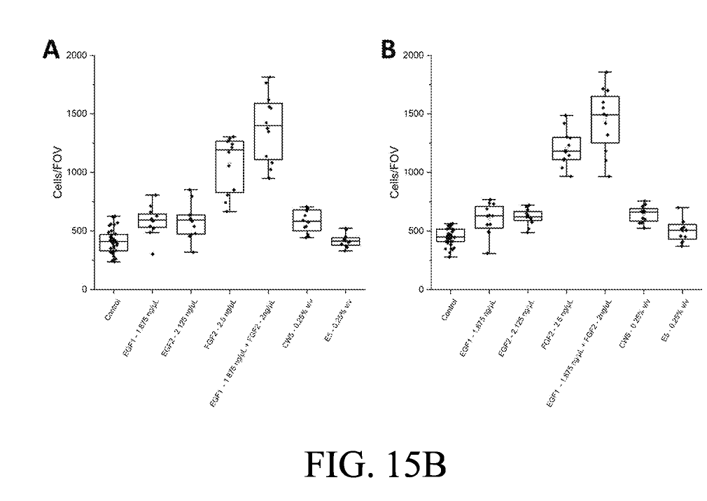

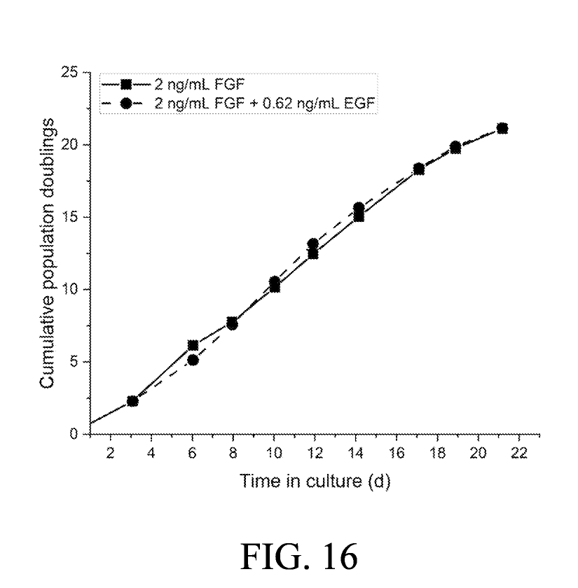

Growth factors were also evaluated. Growth factors are incorporated into isolation media to support cell survival and stabilize the phenotype immediately after enzymatic digestion. During this post-digestion phase, cells experience mechanical and biochemical stress and growth factors can help to maintain viability, promote recovery of membrane integrity and to preserve lineage-specific signaling environments. The supplementation of growth factors to the isolation media can improve the overall quality and functional stability of freshly isolated primary cells. In previous experiments, it was found that a clear positive effect of different growth factors could be shown, such as the addition of EGF1 (0.62 ng/ml) and FGF2 (2ng/ml), which gave superior results regarding cell proliferation (FIG. 15). An appropriate combination of specific growth factors, such as EGF1 and FGF2, can enhance cellular proliferative capacity more than threefold (FIG. 15B) and can effectively improve the cell quality of the isolated cells. It was observed that cells were actively dividing for up to 10 passages (FIG. 16). The experiments showed that concentrations of EGF1 at 0.62 ng/ml and FGF2 at 2ng/ml or only FGF2 at 2ng/ml gave good results (FIG. 15 and 16). Alternatively, these agents can be omitted. However, if this step is omitted it is expected that the yields will be lower.

### Stem Cell Characterization

The stem cells were characterized using the ISCT (International Society for Cellular Therapy) criteria for human stem cells with the addition of porcine specific stem cell markers (such as CD29, CD44, CD90, CD105, CD140, CD146 as positive markers and CD31, CD34, CD45 as negative markers).

In an embodiment, the present invention relates to a method of isolating and cultivating adipose-derived stem cells, the method comprising:

A) removing fat tissue from an area of the body (including the throat area) of an animal (including humans),

B) sterilizing the fat tissue to generate sterilized fat tissue and rinsing the sterilized fat tissue with an aqueous salt solution and place it in a first buffer,

C) transporting the sterilized tissue to a lab in an insulated container,

D) excising non-fat tissue from the sterilized fat tissue to generate pure fat tissue,

E) mincing the pure fat tissue to generate minced fat tissue,

F) adding digestion buffer to the minced fat tissue to generate a digested fat tissue mixture,

G) incubating the digested fat tissue,

H) adding buffer to the digested fat tissue mixture and centrifuging the mixture one or more times to separate the stem cells from the mixture,

I) separating the solid fat fraction from the liquid fraction as well as the cell pellet,

J) centrifuging the solid fat fractions one or more times with the addition of an aqueous salt solution to generate further cell pellets of cells previously still maintained in the fat fraction,

K) centrifuging the liquid fractions one or more times with the addition of an aqueous salt solution to generate further cell pellets of cells previously still maintained in the liquid fraction

L) combining the cell pellets from the aliquots of the initial centrifuged fat tissue, and the processed solid fat fractions as well as the processed liquid fractions.

M) adding erythrocyte lysis buffer to the combined pellets to lyse erythrocytes,

N) filtering the cells through cell strainers to exclude remaining impurities and undigested tissue and to generate isolated stem cells

O) incubating the stem cells to cultivate the stem cells.

In a variation, the animal is a pig. Alternatively, the animal may be a cow, a horse, a goat, a sheep, or another mammal. In a variation, the first buffer and/or the second buffer is transportation buffer. In a variation, the incubating step is performed at 30-45^{o}C, or alternatively, at about 39^{o}C. In a variation, the method further comprises a step of cryogenically preserving the stem cells. In a variation, one or more of ascorbic acid, antibiotics, antimycotics, glucose, growth factors, amphiphilic agents, or glycerol derivatives such as glycerol monostearate, or oxygen are incorporated into the method. In a variation, all of ascorbic acid, antibiotics, antimycotics, glucose, growth factors, amphiphilic agents, such as glycerol monostearate, and oxygen are incorporated into the method.

In a variation, the method is performed under normoxic or hypoxic conditions. In a variation, the method is performed under normoxic conditions. In a variation, the centrifuging steps are performed at 300 g to 500 g for 2-10 minutes. In a variation, tissue was excised from the animal post mortem. In a variation, the transportation step is less than one hour. In a variation, glycerol monostearate is present in the digestion buffer and a concentration of glycerol monostearate is between 0.001% to 1% (w/w) of glycerol monostearate. In a variation, calcium ions are added to the method by adding calcium chloride. In a variation, the incubating step is performed with shaking using a rotary motion shaker at 10-300 rpm. In a variation, an antibiotic cocktail is used in the method. In a variation, an antibiotic cocktail is used in steps B-G.

In an embodiment, the present invention relates to isolated adipose-derived stem cells by the process of:

a) removing fat tissue from the throat area of an animal,

b) sterilizing the fat tissue to generate sterilized fat tissue and rinsing the sterilized fat tissue with an aqueous salt solution and place it in a first buffer,

c) transporting the sterilized tissue to a lab in a pre-cooled buffer and insulated container,

d) excising non-fat tissue from the sterilized fat tissue to generate pure fat tissue,

e) mincing the pure fat tissue to generate minced fat tissue,

f) adding digestion buffer to the minced fat tissue to generate a digested fat tissue mixture,

g) incubating the digested fat tissue

h) adding buffer to the digested fat tissue mixture and centrifuging the mixture one or more times to separate the stem cells from the mixture,

i) separating the solid fat fraction from the liquid fraction as well as the cell pellet,

j) centrifuging the solid fat fractions one or more times with the addition of an aqueous salt solution to generate further cell pellets of cells previously still maintained in the fat fraction

k) centrifuging the liquid fractions one or more times with the addition of an aqueous salt solution buffer to generate further cell pellets of cells previously still maintained in the liquid fraction

l) combining the cell pellets from the aliquots of the initial centrifuged fat tissue, and the processed solid fat fractions as well as the processed liquid fractions.

m) adding erythrocyte lysis buffer to the combined pellets to lyse erythrocytes,

n) filtering the cells through cell strainers to exclude remaining impurities and undigested tissue and to generate isolated stem cells.

o) incubating the stem cells to cultivate the stem cells.

In a variation, erythrocyte removal is performed chemically or physically. In a variation, cell isolation excludes mechanical dissociation beyond mincing. In a variation, digestion occurs without harsh shear forces exceeding 30 rpm.

In a variation, the animal is a pig. In a variation, the process further involves a step of cryogenically preserving the stem cells.

In an embodiment, the present invention relates to a method for optimizing adipose-derived stem cell isolation, comprising adjusting one or more of the following factors: enzymatic composition, chemical composition, amphiphilic agent concentration, oxygen concentration, or nutrient availability, to maximize viable stem cell yield.

In an embodiment, the present invention relates to isolated adipose-derived stem cells that have one or more of the following properties:

(a) a population doubling capacity of at least 10 doublings;

(b) retention of trilineage differentiation potential after minimal five passages; and

(c) viability sufficient to enable expansion into a master cell bank,

wherein the stem cells are suitable for use in cell-cultivated meat production, medical ADSCs or cosmetic stem cells.

In an embodiment, the present invention relates to a method 1 for isolating adipose-derived stem cells from adipose tissue, comprising:

(a) enzymatically digesting adipose tissue using at least one collagen-degrading enzyme in the presence of

(i) calcium ions, and

(ii) an amphiphilic agent effective to increase enzymatic access to hydrophobic extracellular matrix components of the adipose tissue;

(b) mechanically agitating the adipose tissue during digestion under controlled conditions sufficient to liberate viable stem cells while limiting shear-induced cell damage; and

(c) separating liberated cells from undigested tissue and fat fractions to obtain isolated adipose-derived stem cells,

wherein the method produces a stem cell yield of at least 1,000,000, or alternatively, 1,500,000 viable cells per gram of adipose tissue within 24 hours of digestion.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A method of isolating and cultivating adipose-derived stem cells, the method comprises:
A) excising fat tissue from an animal,
B) sterilizing the fat tissue to generate sterilized fat tissue and rinsing the sterilized fat tissue with an aqueous salt solution and place it in a first buffer,
C) transporting the sterilized tissue to a lab in an insulated container,
D) excising non-fat tissue from the sterilized fat tissue to generate pure fat tissue,
E) mincing the pure fat tissue to generate minced fat tissue,
F) adding digestion buffer to the minced fat tissue to generate digested fat tissue mixture,
G) incubating the digested fat tissue
H) adding buffer to the digested fat tissue mixture and centrifuging the mixture one or more times to separate the stem cells from the mixture,
I) separating the solid fat from the liquid fraction as well as the cell pellet,
J) centrifuging the solid fat fractions one or more times with the addition of an aqueous salt solution buffer to generate further cell pellets of cells previously still maintained in the fat fraction
K) centrifuging the liquid fractions one or more times with the addition of an aqueous salt solution to generate further cell pellets of cells previously still maintained in the liquid fraction
L) combining the cell pellets from the aliquots of the initial centrifuged fat tissue, and the processed solid fat fractions as well as the processed liquid fractions,
M) adding erythrocyte lysis buffer to the combined pellets to lyse erythrocytes,
N) centrifuging and discarding the second liquid and combining the pellets of erythrocyte lysis buffer - treated stem cells,
O) resuspending cell pellets in isolation media
P) filtering the cells through cell strainers to exclude remaining impurities and undigested tissue and to generate isolated stem cells, and
Q) incubating the stem cells to cultivate the stem cells.

2. The method of claim 1, wherein the method further comprises utilizing the adipose-derived stem cells for cultivated meat.

3. The method of claim 1, wherein the first buffer and/or the second buffer is an aqueous salt solution and/or transportation buffer, and the first and/or the second buffer optionally contains antibiotics and/or antimycotics and a minimum yield is 0.5 X 10⁶ cells per gram of tissue in a 24 hour period or less.

4. The method of claim 1, wherein the animal is a pig.

5. The method of claim 1, further comprises a step of cryogenically preserving the stem cells.

6. The method of claim 1, wherein one or more of antioxidants, antibiotics, antimycotics, glucose, growth factors, food grade emulsifiers and/or amphiphilic agents, or oxygen are incorporated into the method, and
wherein the antioxidants comprise ascorbic acid, the food grade emulsifiers and amphiphilic agents comprise glycerol monostearate, and all of ascorbic acid, antibiotics, antimycotics, glucose, growth factors, glycerol monostearate, and oxygen are incorporated into the method.

7. The method of claim 1, further comprising enzymatically digesting adipose tissue in the presence of
(i) a collagen-degrading enzyme,
(ii) an amphiphilic agent that increases enzyme access to a hydrophobic extracellular matrix, and
(iii) calcium ions,
under conditions that maximize viable stem cell yield.

8. The method of claim 1, wherein the method is performed under normoxia or hypoxia conditions.

9. The method of claim 1, wherein the method is performed under normoxia conditions and a yield is at least 0.5 X 10⁶ cells in a 24 hour period or less.

10. The method of claim 1, wherein the centrifuging steps are performed at 50 to 600 g for 0.5-15 minutes.

11. The method of claim 1, wherein the tissue samples from the animal are taken post mortem.

12. The method of claim 1, wherein the tissue samples from the animal are taken pre mortem and/or the transporting step is less than one hour.

13. The method of claim 7, wherein a food grade emulsifier or amphiphilic agent such as glycerol monostearate is in the digestion buffer and a concentration of glycerol monostearate is between 0.0001% to 10% (w/w).

14. The method of claim 1, wherein calcium ions are added by adding calcium chloride or other calcium salts.

15. The method of claim 1, wherein the incubating step is performed with shaking using a rotary motion shaker between 1 rpm and 400 rpm.

16. The method of claim 1, wherein an antibiotic cocktail is used in the method.

17. The method of claim 16, wherein the antibiotic cocktail is used in steps B-G.

18. Isolated stem cells derived from adipose tissue, the isolated stem cells isolated by the process of:
a) removing fat tissue from the throat area of an animal,
b) sterilizing the fat tissue to generate sterilized fat tissue and rinsing the sterilized fat tissue with aqueous salt solution or a physiological buffer and placing it in a first buffer,
c) transporting the sterilized tissue to a lab in an insulated container,
d) excising non-fat tissue from the sterilized fat tissue to generate pure fat tissue,
e) mincing the pure fat tissue to generate minced fat tissue,
f) adding digestion buffer to the minced fat tissue to generate a digested fat tissue mixture,
g) incubating the digested fat tissue mixture,
h) adding buffer to the digested fat tissue mixture and centrifuging the digested fat tissue mixture one or more times to separate the stem cells in the digested fat tissue mixture and a liquid,
i) separating a solid fat fraction from the liquid as well as a cell pellet,
j) centrifuging the solid fat fraction one or more times with the addition of a first PBS buffer to generate further cell pellets of cells previously still maintained in the fat fraction,
k) centrifuging the liquid fractions one or more times with the addition of a second PBS buffer to generate further cell pellets of cells previously still maintained in the liquid fraction,
l) combining the cell pellets from the aliquots of the initial centrifuged fat tissue, and the processed solid fat fractions as well as the processed liquid fractions,
m) adding erythrocyte lysis buffer to the combined pellets to lyse erythrocytes to generate erythrocyte lysis buffer - treated stem cells and a second liquid, centrifuging and discarding the second liquid and combining cell pellets of the erythrocyte lysis buffer - treated stem cells,
n )resuspending the cell pellets in an isolation media to generate suspended cells,
o )filtering the suspended cells through cell strainers to remove remaining impurities and undigested tissue and to generate isolated stem cells, and
p) incubating the isolated stem cells to cultivate the stem cells, and
q) wherein the process optionally involves a step of cryogenically preserving the stem cells.

19. The isolated stem cells of claim 18, wherein the animal is a pig.

20. A method for isolating adipose-derived stem cells from adipose tissue, the method comprising:
(a) enzymatically digesting adipose tissue using at least one collagen-degrading enzyme in the presence of
(i) calcium ions, and
(ii) an amphiphilic agent effective at increasing enzymatic access to hydrophobic extracellular matrix components of the adipose tissue;
(b) mechanically agitating the adipose tissue during digestion under controlled conditions sufficient to liberate viable stem cells while limiting shear-induced cell damage; and
(c) separating liberated cells from undigested tissue and fat fractions to obtain isolated adipose-derived stem cells,
wherein the method produces a stem cell yield of at least 1,000,000 viable cells per gram of adipose tissue within 24 hours of digestion.
